(19) United States
(12) Patent Application Publication
(10) Pub. No.: US 2004/0152974 A1
(43) Pub. Date: Aug. 5, 2004

(54) Cardiology mapping and navigation system

(76) Inventor: Stephen Solomon, Baltimore, MD (US)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/764,026

(22) Filed: Jan. 23, 2004

Related U.S. Application Data
(63) Continuation-in-part of application No. 10/116,853, filed on Apr. 5, 2002.
(60) Provisional application No. 60/442,018, filed on Jan. 23, 2003.
(60) Provisional application No. 60/282,260, filed on Apr. 6, 2001.

(51) Int. Cl. A61B 5/05

(52) U.S. Cl. 600/424; 600/509

(57) ABSTRACT

A method and apparatus are provided for superimposing the position and orientation of a diagnostic and/or treatment device on a previously acquired three-dimensional anatomic image such as a CT or MRI image, so as to enable navigation of the diagnostic and/or treatment device to a desired location. A plurality of previously acquired three-dimensional images may be utilized to form a “movie” of the beating heart which can be synchronized with a patient's EKG in the operating room, and the position of the diagnostic and/or treatment device can be superimposed on the synchronized “movie” of the beating heart. An electrophysiological map of the heart can also be superimposed on the previously acquired three-dimensional antaomic image and/or the “movie” of the beating heart.

This application claims the benefit of U.S. Provisional Application No. 60/442,018 filed Jan. 23, 2003 which is a continuation in part of U.S. application Ser. No. 10/116,853 filed Apr. 5, 2002 which in turn claims the benefit of U.S. Provisional Application No. 60/282,260 filed Apr. 6, 2001, the entire contents of each of which are incorporated herein by reference.

### BACKGROUND OF THE INVENTION

Cardiologists use catheters in the heart to acquire diagnostic information (either injecting dye for angiograms or sensing electrical information). They also may use devices such as radiofrequency ablation catheters to deliver therapy to the heart. These diagnostic and treatment devices are typically maneuvered in the heart based on an x-ray fluoroscopic image. This often results in fluoroscopy times of one hour or more during prolonged electrophysiological procedures, and results in a substantial radiation exposure for both the patient and cardiologist, especially when considering the frequent need for repeat procedures. In addition, the heart is a three dimensional structure whereas the fluoroscopic image is only two dimensional. And since knowing the exact anatomic location of a diagnostic or treatment device in the heart is extremely important in order to acquire accurate diagnostic information or to accurately deliver a therapy to particular locations in the heart, the conventional use of fluoroscopic images is often inadequate.

One particular area in which knowing the anatomic position of cardiac catheters would be particularly helpful is electrophysiology, and one particular application for this is in the treatment of paroxysmal atrial fibrillation stemming from the pulmonary veins. In 1998 Haissaguerre et al. (The New England Journal of Medicine, Sep. 3, 1998) reported that the pulmonary veins were the source of the majority of cases of paroxysmal atrial fibrillation and that by ablating the pulmonary vein foci, patients could be successfully treated. Since that time a number of studies have verified this notion and a better understanding has evolved. It is now believed that the best location for ablating pulmonary veins is the ostium, that is, the junction between left atrium and pulmonary veins.

A number of methods using a variety of energy sources have evolved to treat the ostia of the pulmonary veins. Some take an anatomic approach and simply ablate circumferentially around the pulmonary veins; others prefer to map the electrical rhythms and focally ablate at the ostia.

Recently, Haissaguere et al. (Circulation, Mar. 28, 2000) have developed a method of mapping the pulomonary ostia with a “lasso” catheter. The lasso catheter contains a plurality of electrodes which independently map the electrical activity of adjacent tissue. A separate, standard radiofrequency ablation catheter is then used to focally ablate the tissue at one or more of the plurality of electrodes which indicate an abnormal rhythm.

One of the major challenges in performing this procedure is that the standard use of two dimensional fluoroscopy does not reveal the necessary anatomic information to identify the location of the pulmonary veins. In particular, it is difficult to know exactly where the ostia are located. Even with use of radiographic contrast, the two dimensional image produced by fluoroscopy is inadequate. Furthermore, visualizing the essentially two-dimensional lasso catheter in the three dimensional space of the heart is confusing. Thus, as shown in FIG. 5, it is difficult to know the exact location and orientation of the lasso catheter. Specifically, it is difficult to know whether the loop of the lasso is coming out at the viewer or back in to the image. Still further, it is also difficult to move the ablation catheter (identified by a pentagon pointer in FIG. 5) to the particular desired electrode of the lasso catheter that indicates an abnormal signal. This is a three dimensional process in two dimensions. Biplane fluoroscopy can help, but is not perfect.

Another problem for cardiologists is that the pulmonary veins are not consistent person to person. Such anatomic variability complicates the procedure. To counter this, most electrophysiologists who perform ablation procedures on the pulmonary veins now require cross-sectional imaging (CT or MRI) to help them identify the pulmonary vein anatomy. Conventionally, however, such CT or MRI images are independently viewed by the electrophysiologist during performance of the procedure. That is, such CT or MRI images are conventionally used as a separate source of anatomical information, without being positionally coordinated with the procedure being performed.

Recently, position sensors have been used to provide navigational information based on previously acquired CT or MRI image in surgery. The previously acquired CT or MRI image are brought to the operating room on computer. Then, the position of a pointer or surgical instrument inserted in the patient is registered with the previously acquired CT or MRI image in the operating room. The position of the pointer or surgical instrument is then tracked either by electromagnetic fields, ultrasound, optics, or mechanical joints. Thus, the position and orientation of the instrument can be continually displayed on the previously acquired images. This information is then used to help guide the physician. In particular, such navigational tracking techniques have been used in brain surgery (See Solomon SB, Interactive images in the operating room, J Endourol 1999; 13:471-475.)

Position sensors are also commonly used to produce electrophysiological maps of the heart based on detected electrical and mechanical information of the heart (i.e., using a diagnostic electrode catheter sold by Biosense-Webster). This allows for identification of the source for electrical arrhythmias and allows the physician to move an ablation catheter to an abnormal arrhythmogenic focus. Conventionally, however, these electrical maps do not use previously acquired anatomic image data. Instead, position sensors are merely used to create a computer generated “cartoon” image by touching the walls of the heart and recording electrical activity. Such a computer generated electrophysiological map is shown in FIG. 6. The electrophysiological map shown in FIG. 6 is utilized for detecting abnormal electrical activity. But the electro-physiological map shown in FIG. 6 does not supply sufficient anatomic detail to optimally perform many catheter based procedures. It also does not show the branching patterns of the veins, and it does not show the proximity of a lasso catheter to an ablation catheter.

One point to note is that the previously acquired image utilized in conventional navigational tracking techniques are taken at one particular point in time. In terms of brain surgery, for example, the use of such a single previously acquired image is adequate because the position of the head is fixed and there is little movement of the anatomy being operated on.

However, the heart is a beating organ that actually moves inside the body of the patient during performance of a procedure. This makes it even more difficult to know the precise anatomic location of a diagnostic or treatment device within the heart at any given moment in time.

### SUMMARY OF THE INVENTION

In order to more accurately enable a physician to navigate a diagnostic and/or treatment device in the heart, the present invention provides a method and apparatus for superimposing the position and orientation of the diagnostic and/or treatment device on a previously acquired image such as a CT or MRI image. This couples the ability to see the anatomy of the heart such as the pulmonary veins and their anatomic variations from a patient specific CT or MRI image with the ability to track the diagnostic and/or treatment device in real-time so as to enable navigation of the diagnostic and/or treatment device to a desired location. At the same time, this technique reduces the conventional reliance on x-ray fluoroscopy and thereby decreases radiation exposure

In addition, according to the present invention, a “loop” of previously acquired CT or MRI images encompassing an entire cardiac cycle can be utilized to form a “movie” of the beating heart. This beating heart movie can then be synchronized with the patient's EKG in the operating room or synchronized with a reference catheter attached to the heart wall. In this latter case the reference catheter position will immediately indicate the phase in the cycle of the “movie” of the beating heart. With the use of such a synchronized beating heart movie as a “road map”, the cardiologist will be enabled to know the exact anatomic location of the inserted diagnostic and/or treatment device at all times during each phase of the cardiac cycle. And it is noted that the beating heart movie can be controlled so that when the patient's heart rate increases or slows, as detected by the EKG, the movie can be sped up or slowed in a corresponding manner.

Still further, the present invention also provides a method and apparatus for superimposing a computer generated electrophysiological map of the heart on a previously acquired CT or MRI image so that the electrical activity of the heart can be viewed in relation to the true anatomic structure of the heart.

### BRIEF DESCRIPTION OF THE DRAWINGS

FIG. 1A is a schematic drawing of the standard anatomy of the heart.

FIG. 1B is an image from a three dimensional dataset of a gadolinium enhanced cardiac MRI. The image is in an essentially coronal plane depicting the left atrium (LA) and pulmonary veins (PV).

FIG. 1C is an axial image of the heart from a cardiac MRI. The left atrium (LA) and pulmonary veins (PV) are shown.

FIG. 2A is a schematic drawing of a diagnostic electrophysiology lasso catheter having a plurality of electrodes which are each able to record subjacent electrical activity. As shown in FIG. 2A, a plurality of position sensors are provided on the tip of the lasso catheter.

FIG. 2B is a schematic drawing of an ablation catheter having a position sensor provided on a tip thereof.

FIG. 3 is a schematic drawing of the left atrium with a lasso catheter in the left superior pulmonary vein. The ablation catheter is also depicted.

FIG. 4 is a schematic drawing of the monitor showing the previously acquired CT or MRI image of the heart with superimposed indicators of the position of the ablation catheter and the lasso catheter. Multiple indicators are shown for the lasso catheter corresponding to respective sensing elements thereof. Below the anatomic image is a navigator view showing the distance and orientation of the ablation catheter to direct the user to the desired electrode of the lasso catheter.

FIG. 5 is a typical AP fluoroscopic image of the chest depicting the lasso catheter (arrow) presumably in a pulmonary vein. This two dimensional image shows little three dimensional anatomic detail.

FIG. 6 is a typical computer generated (Carto, Biosense-Webster) electro-physiological map of the heart.

FIGS. 7A, 7B, 7C, and 7D show a CT of the heart in coronal, sagital, axial, and 3-D views, respectively, with electrophysiology information superimposed thereon.

### DETAILED DESCRIPTION

The present invention will be described in detail below in particular connection with the treatment atrial fibrillation at the ostia of the pulmonary veins utilizing an electrophysiology diagnostic lasso catheter and an ablation catheter.

However, the navigation technique of the present invention is equally applicable to numerous other cardiology procedures. In particular, other clinical applications to which the present invention is equally applicable include: (i) electrophysiologic ablations of other dysrhythmias such as sources of ventricular tacchycardia, (ii) stent placement at identified stenoses and guided by functional nuclear medicine images indicating infarcted or ischemic tissue, (iii) percutaneous bypass procedures going for instance, from the aorta to the coronary sinus, (iv) injection of angiogenesis factors or genes or myocardial revascularization techniques delivered to particular ischemic walls noted by nuclear images or wall thickness, and (v) valvular procedures. Indeed, the present invention is applicable to any diagnostic or treatment operation performed in the heart which relies upon exact positioning within the heart.

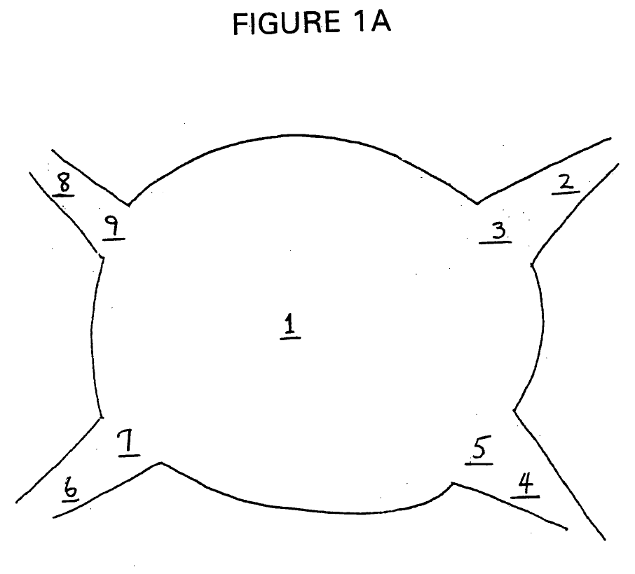

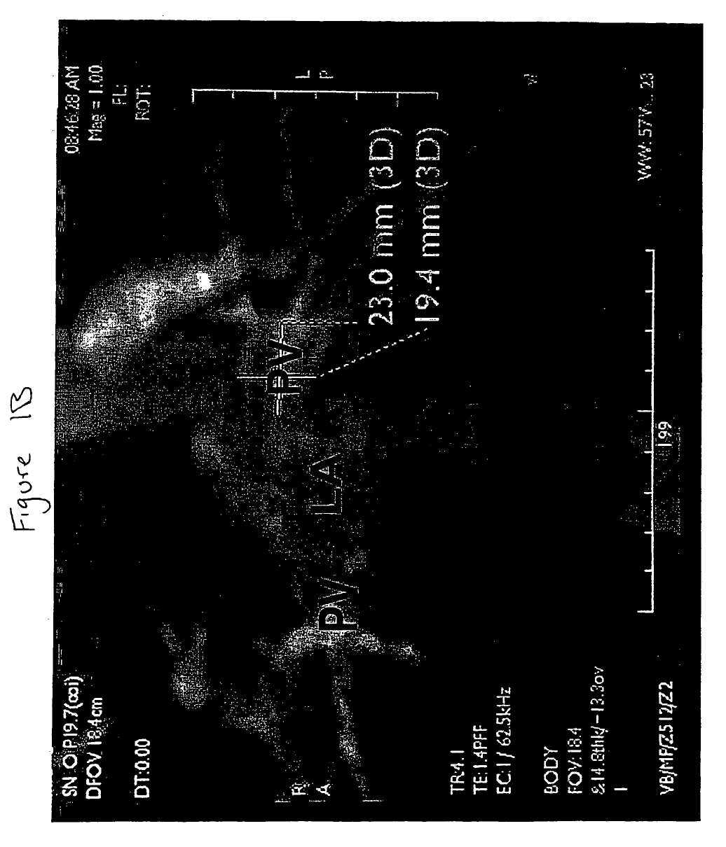

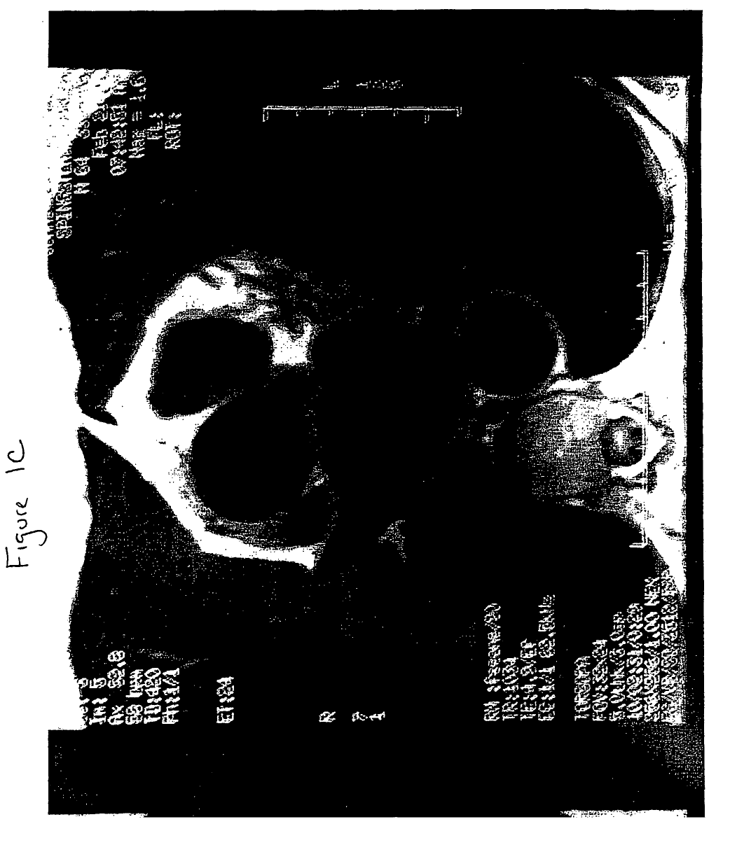

FIG. 1A is a schematic drawing of the standard anatomy of the heart, wherein reference numeral 1 identifies the left atrium, reference numeral 2 identifies the left superior pulmonary vein, reference numeral 3 identifies the ostium of the left superior pulmonary vein, reference numeral 4 identifies the left inferior pulmonary vein, reference numeral 5 identifies the ostium of the left inferior pulmonary vein, reference numeral 6 identifies the right inferior pulmonary vein, reference numeral 7 identifies the ostium of the right inferior pulmonary vein, reference numeral 8 identifies the right superior pulmonary vein, and reference numeral 9 identifies ostium of the right superior pulmonary vein.

Previous Imaging

A CT, MR, nuclear medicine or ultrasound image is acquired for use as a “roadmap” for performing a cardiology procedure. For example, the MR images shown in FIGS. 1B and 1C may be utilized as the “roadmap”. However, any image showing the detailed anatomy of the heart can be used as the “roadmap”.

The “roadmap” image may be acquired at any time prior to the procedure to be performed. However, the image should preferably be acquired within 24 hours of the procedure.

According to a preferred embodiment of the present invention, a series of images may be taken with cardiac gating. The series of images can then be sorted and processed using a standard software package such as a standard GE (General Electric Medical Systems, Milwaukee, Wis.) cardiac MRI software package to produce a “movie” or “cine” of the beating heart. Image information acquired during contraction is kept separate from image information acquired during relaxation. This allows the reconstruction of the images in a “movie” or “cine” fashion. And the movie or cine can then be synchronized to the patient's actual EKG cycle in the operating room during performance of the procedure.

During the image acquisition fiducial markers may be placed on the patient's chest. These markers are kept on the chest until after the cardiac procedure. These markers may be stickers which will appear in the image or images and allow the patient to be aligned consistently later in the operating room.

The acquired image or images are then electronically transmitted to a computer, and a display device is provided in the operating room on which they may be viewed.

Registration

In the operating room, the patient is registered with the previously acquired image or images.

Several methods of registration exist. One method is to use the fiducial markers which may be provided on the patient. Each marker is touched with a position sensor in the operating room. While touching the marker, the position of the marker with respect to the previously acquired image or images is ascertained by the computer in which the previously acquired image or images have been loaded. The touching of several markers will enable image registration to be achieved.

An alternative registration method that does not involve external fiducial markers is to touch several points with a position sensor of a catheter within the patient's heart. The several points then define a computer shape. And by coordinating the defined shape with the previously acquired image or images, the computer can perform image registration. Ideally, this position sensor will be acquiring coordinates for the registration in a gated fashion with the cardiac cycle. The several points which are touched with the position sensor may be fluoroscopic landmarks which are confirmed by MR. The landmarks may be, for example, the left ventricular apex, coronary sinus or valve plain.

Electrical landmarks that correspond to known anatomic positions within the heart may also be touched with a position sensor to achieve image registration. According to this method of registration, for example, the atrioventricular node (AV node) may be located electrically and the anatomic position of the AV node, the interatrial septum near the tricuspid valve, may be indicated on the MR.

Still further, variations in pressure within the heart may be utilized to register the image of the heart. In this method of registration, for example, the location at which pressure changes between the right atrium and right ventricle is located to indicate a position near the tricuspid on the MR image.

Tracking

Several position sensing systems are possible; some use electromagnetic fields while others use ultrasound. According to one embodiment of the present invention described below, electromagnetic fields are used.

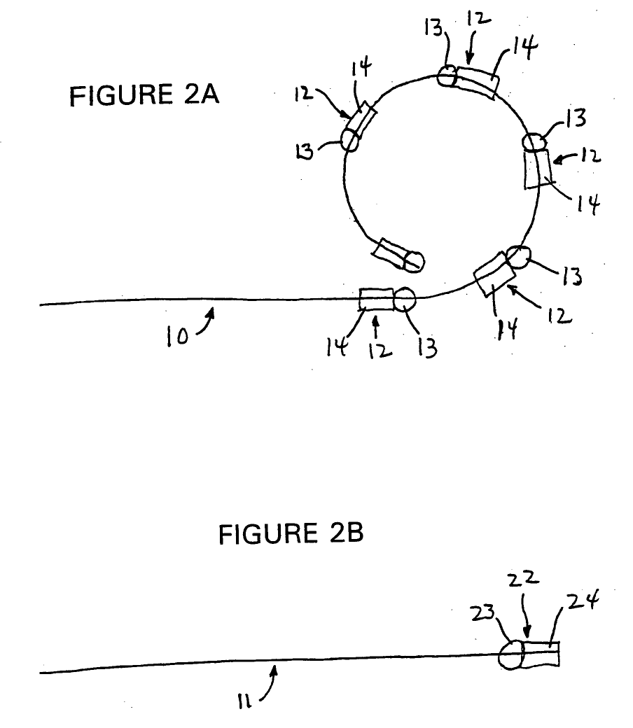

As shown in FIGS. 2A and 2B, respectively, six position sensors 12 are provided along the distal portion of the lasso catheter 10, and one position sensor 22 is provided at the tip of the ablation catheter 11. The position sensors 12 of the lasso catheter 10 each comprise a coil 13, and an electrode 14 for performing sensing. The position sensor 22 of the ablation catheter 11 comprises a coil 23 and an electrode 24 for performing ablation. The coils 13 and 23 may each comprise three miniature orthogonal coils, and the electrodes 14 and 24 may each be adapted for sensing and/or ablation operations. Each of the position sensors 12 and 22, moreover, is individiually identifiable, either by being separately wired or by including indiviually identifiable markers or signal characteristics.

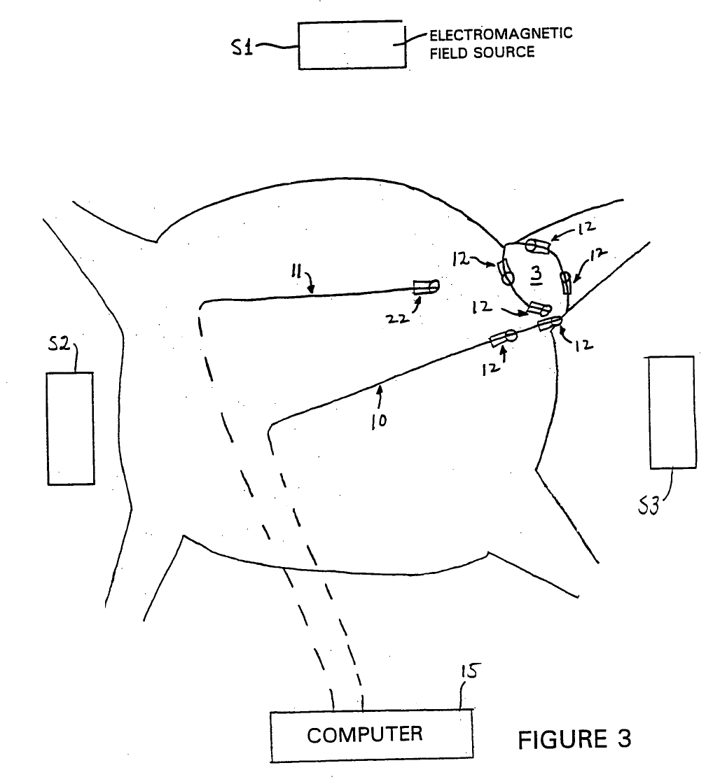

As shown in FIG. 3, the lasso catheter 10 is inserted into the heart and is placed, for example, in the vicinity of the ostium 3 of the superior left pulmonary vein 2.

In the operating room, a plurality (for example, three) electromagnetic field sources S1, S2 and S3 with distinct frequency and/or amplitude are placed external to the patient.

Then, when the external electromagnetic field sources S1, S2 and S3 are activated, the coils 13 and 23 of the position sensors 12 and 22 act as receivers and transmit information on distance and orientation to a computer 15.

The computer 15 then calculates the position and orientation of the coils 13 and 23 of the position sensors 12 and 22, so that the exact location and orientation of the lasso catheter 10 and ablation catheter 11 can be determined.

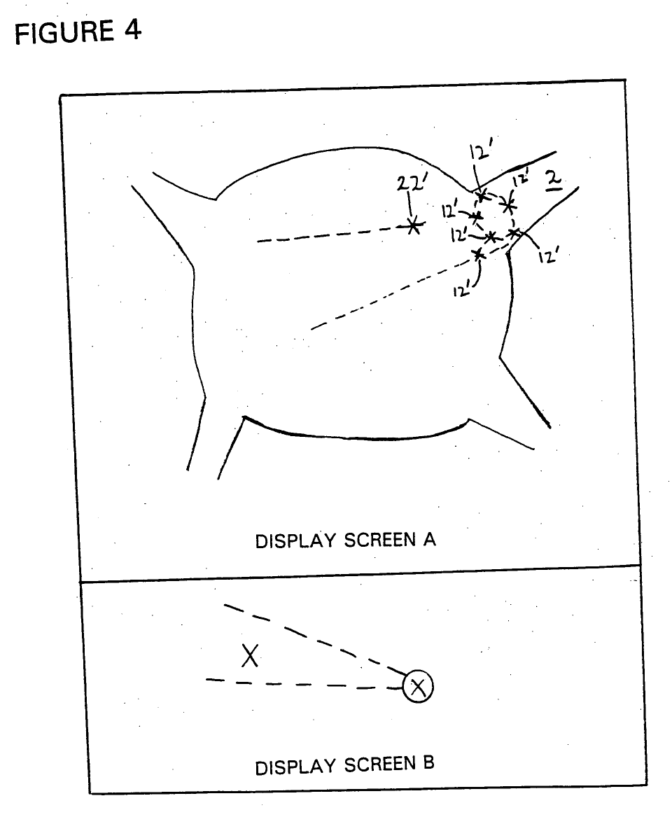

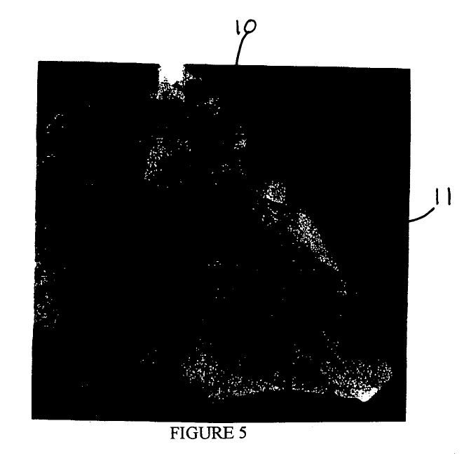

As shown in on Display Screen A in FIG. 4, indicator 22′ shows the position of the position sensor 22 at the tip of the ablation catheter 11, and indicators 12′ show the position of the position sensors 12 of the lasso catheter 10. Thus, the position of each of the lasso catheter 10 and ablation catheter 11 can be displayed in a superimposed manner on the previously acquired image or images, so that the physician can ascertain the true anatomical position of the lasso catheter 10 and ablation catheter 11 in the heart. This will allow the physician to guide the lasso catheter to the ostia seen on the anatomic MR images.

As the physician moves the lasso catheter 10 in the heart, the indicators 22′ move in a corresponding manner on the previously acquired MRI roadmap image. The physician is thus able to visualize the position of the lasso catheter 10 on the MR image as it is moved within the heart. The lasso catheter 10 can thus be brought to the anatomically desired location at the desired ostium 3. And since the lasso catheter 10 is in three dimensional space, the indicators 12′ of the multiple position sensors 12 provided at the distal end of the lasso catheter 10 can indicate the orientation of the ring of the lasso catheter 10 in the three dimensional space of the heart. The ring can be superimposed on the three dimensional CT or MR images, and the images can be moved to show the ring sitting in the desired ostial location.

It is noted that in the example described above, multiple position sensors 12 are provided on the single lasso catheter 10. This enables visualization of the complex and realistic positioning and twisting of the catheter and lasso coil thereof.

Once the lasso catheter 10 is accurately positioned at the desired ostium 3, diagnostic electrical information is acquired from each individual electrode 14 provided on the lasso catheter 10. This information is used to determine the exact location on the ostium at which ablation is to be performed.

The tip of the ablation catheter 11 is then guided to the exact electrode 14 of the lasso catheter 10 to the position in the heart that requires ablation. This is achieved using the indicator 22′ indicating the position of the position sensor 12 at the tip of the ablation catheter 11 which is superimposed in a moving manner on the previously acquired MRI roadmap image.

Thus, since the positions of the diagnostic catheter 10 and the ablation catheter 11 are both known, the computer can calculate a distance from one to the other. And as shown in Display Screen B in FIG. 4, an “Airplane type Distance Navigation” can be utilized to guide the ablation catheter 11 to the desired senesor 12 of the lasso catheter 10 using the indicator 22′ and the desired one of the indicators 12′.

While in the procedure room, the physician will have the navigation computer with CT or MR images to guide the procedure. He/she will also still have the real time fluoroscopic images which can act as confirmation of the general position and status of the catheters. This might be important, for instance, if the shaft of the lasso catheter 10 were bending while the ring stayed intact.

One particularly interesting aspect of the present invention is that a series of previously acquired CT or MRI images can be acquired to produce a “movie” or “cine” of the beating heart. Such a series of images can then be gated to an EKG and synchronized with a real time EKG to produce a real-time “beating” image of the heart in the operating room. Thus, when the patient's heart rate increases or slows, as detected by the EKG, the movie or cine can be sped up or slowed in a corresponding manner. And with the use of such a synchronized “beating heart” movie or cine as a “road map”, the physician will be enabled to know the exact anatomic location of the inserted diagnostic and/or treatment device at all times during each phase of the cardiac cycle.

In particular, it is noted that since the position of a catheter is fixed in space inside the patient's heart, the distance from the cardiac wall varies with the beating of the patient's heart. Conventional cardiology techniques do not take such distance variation due to the beating of the heart into account. In fact, using conventional navigation techniques, the distance from a catheter to the cardiac wall artificially appears to be constant. However, by utilizing a synchronized “beating heart” movie or cine as a “road map” according to the technique of the present invention, the distance variation caused by beating of the heart can be taken into account. Still further, the use of such a “beating heart” movie or cine may allow the timing of therapeutic application to be synchronized with the beating of the patient's heart. For example, the timing at which ablation is performed may be synchronized to be effected during contraction when coronary blood flow is limited as opposed to during relaxation when blood flow is maximal.

Another facet of the invention is to enable a faster and more accurate way of registering previously acquired MRI or CT images with the actual beating heart. Namely, a position sensor is touched to the wall of the heart so that it will move with the heart wall throughout the beating heart cycle. Positional coordinates of the sensor are collected with each beat to define a beating structure. This beating structure can then be computer fitted to a “movie” or “cine” of the beating heart created based on the previously acquired MRI or CT images of the heart. For greater registration accuracy, the positional information gathered during a heart beat can be repeated at a plurality of points on the heart wall.

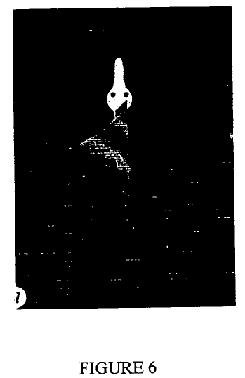

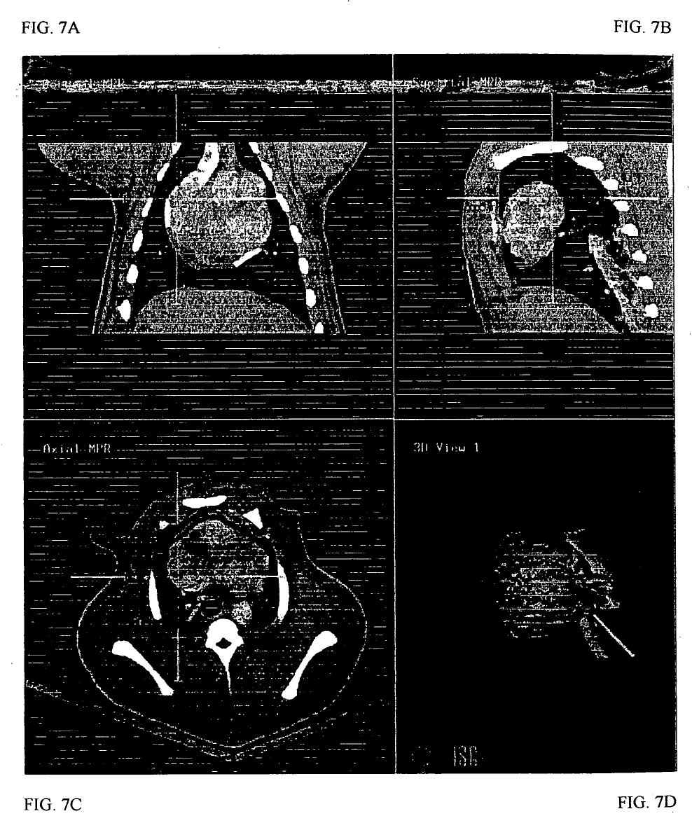

Still further, it is noted that the cardiological mapping and navigation technique of the present invention can also be utilized in conjunction with known electrophysiological mapping techniques. Namely, a standard electrophysiology mapping electrode catheter (such as the diagnostic electrode catheter sold by Biosense-Webster) may be utilized to obtain electrical information at various detected positions on the wall of the heart, and this information can then be utilized to produce an electrical map of the heart such as the one shown in FIG. 6. Such an electrophysiological map of the heart can then be superimposed on the previously acquired MRI or other roadmap image in order to produce an actual anatomical image showing current electrical activity, as shown in FIGS. 7A-7D. That is, the technique of the present invention is carried out as described above, except that at any desired time, the physician can additionally superimpose the electrophysiological map of the heart on the previously acquired still or “movie” roadmap image of the heart, as desired.

FIGS. 7A, 7B, 7C, and 7D show a CT of the heart in coronal, sagital, axial, and three-dimensional views, respectively. The yellow cross-hairs indicate the position of the tip of the catheter, and the yellow/red/green coloring superimposed on the CT images represent electrophysiology information gathered during the procedure. This superimposed coloring represents the timing of activation of the electrical signals of the heart.

Thus, the images shown in FIGS. 7A-7D combine both electrophysiological information with anatomic information so that the physician is provided with detailed anatomical information and detailed electrical activity information in a single image. As a result, the propagation of electrical waves can be seen on an actual anatomic image, and such an image can be used to accurately guide a diagnostic and/or treatment device to a desired location to enable improved therapeutic procedures to be performed. For example, a catheter could be guided to the opening of the pulmonary vein for ablation, to a location of wall motion abnormality for injection of genes, and/or to an infarct for treatment of electrical abnormalities.

### EXAMPLE

Animal Preparation

A 50 kg domestic swine was sedated with acepromazine 50 mg IM and ketamine 75 mg IM. Thiopental 75 mg IV were administered prior to intubation. The animal was maintained on inhaled isoflurane 2% in air during the catheter procedure. During transportation to the CT scanner and during scanning the swine was given pentobarbital IV to maintain anesthesia. At the end of the procedure the animal was euthanized using an overdose of IV pentobarbital.

CT Scanning

Prior to scanning nine 1.0 mm metallic nipple marker stickers were placed across the chest of the pig to allow for later registration of the images. The swine was imaged with a spiral CT (Somatom Plus 4, Siemens, Iselin, N.J.) using parameters of 2 mm thick slices, 4 mm/sec table speed, and approximate exam time of 40 seconds. Intravenous iohexol contrast (Omnipaque 350, Nycomed, Buckinghamshire, United Kingdom) 100 ml at a rate of 2 cc/sec was administered just prior to imaging. End expiration breath hold was simulated by turning off the ventilator for approximately 45 seconds during the scan while the pig was paralyzed with pancuronium (0.5 mg/kg IV). The obtained images were then electronically transmitted to the navigation computer in the fluoroscopy suite.

Navigation System

The navigation system (Magellan, Biosense Webster Inc., New Brunswick, N.J.) comprised a computer containing the three-dimensional CT or MR images, and an electromagnetic locator pad that was placed under the patient. This pad generated ultralow magnetic fields (5×10-5 to 5×10-6 T) that coded both temporally and spatially the mapping space around the animal's chest. The locator pad included three electromagnetic field generating coils. These fields decayed with distance allowing the position sensor antenna at the tip of the catheter to identify position in space. Orientation was provided by the presence of three orthogonal antennae in each catheter tip sensor. Previous studies had shown accuracy for in vitro work to be approximately 1 mm. The navigation system relied on two position sensor catheters, the reference catheter and the active procedural catheter. The reference catheter with a position sensor at its tip was taped to the chest of the swine. This supplied additional information about respiratory, positional changes and helped maintain the registered frame of reference. The procedural catheter with a similar position sensor at its tip for tracking its position and orientation was used to navigate within the heart and vascular tree.

Image Registration

The CT images were transmitted to the navigation system computer (Magellan, Biosense) located in the fluoroscopy suite. Three-dimensional reconstructions were made using the relative differences in CT Hounsfield units of the various structures. The procedural catheter was used to touch each of the nine metallic stickers placed across the animal's chest prior to CT. With each sticker the computer cursor was placed over the corresponding marker on the CT image. This allowed the “registration” of the image with the live pig.

Accuracy and Precision Assessment

Repeated measurements as described below of the nine surface markers were performed at the beginning and end of the study and served as a surrogate to estimate accuracy and precision of intracardiac manipulation.

To test accuracy, the procedural catheter was moved to each of the nine markers on the chest. At each marker the distance between the location that the navigation system believed was the location (M) of the marker and the actual location (T) of the marker was determined. The position error was calculated using the following equation:

\sqrt{(Mx−Tx)²+(My−Ty)²+Mz−}Tz)² (Formula 1)

where (Mx, My, Mz) and (Tx, Ty, Tz) are the coordinates of points M and T respectively. Five independent attempts at touching each of the nine markers were performed. Data was averaged and error ranges noted for the nine marker points.

To test the precision of the system, an average point was obtained from the average coordinates of the five independent measurements per marker in three-dimensional space. Distance from each of the five measured points to this virtual point was then measured. Data was averaged and error ranges noted for the nine marker points.

Catheterization and Image Correlation

Right femoral 8F sheaths were placed in both femoral vein and artery. The procedural catheter with the position sensor at its tip was inserted into the femoral vein and then into the femoral artery. Real-time movement of the catheter was observed on the CT images as noted by a cross-hair display. Correlation with biplane fluoroscopic images was observed after positioning the catheter in the right atrium, right/left ventricle and pulmonary artery. However, no fluoroscopic imaging was needed to navigate to these structures.

Accuracy and Precision Assessment

Accuracy measurements were repeated five times per actual marker in three-dimensional space. The distance between the actual marker on the skin and where the computer indicated the tip was located was measured. The average accuracy was determined to be 4.69±1.70 mm. However, in this example, the reference catheter primarily accounted for antero-posterior motion of the chest wall during respiration. This is probably the reason for more error existing in the lateral points for which lateral chest wall motion is the main source of movement. In neglecting the most lateral two points the accuracy measured in this example improved to 3.98±1.04 mm.

Precision measurements were made by measuring the distance between a virtual point representing the three-dimensional average of the five registrations and each of the five registrations. The precision was determined to be 2.22±0.69 mm, and neglecting the most lateral two points the precision was determined to be 2.21±0.78 mm.

Additional advantages and modifications will readily occur to those skilled in the art. For example, although the described embodiment is directed to three dimensional imaging of the heart, two dimensional imaging is also possible. The two dimensional display may be produced substantially in the same manner as the three dimensional display. An EKG synchronized CT, MR, Ultrasound or Nuclear Medicine image is acquired. A positional image of the heart is acquired and also synchronized with the EKG, with multiple positions taken at different points within the cardiac cycle. The acquired image and positional images are registered using the EKG. And the registration is oriented and the appropriate mathematical transformation is created using one of the registration methods described hereinabove.

Various further modifications may be made without departing from the spirit or scope of the general inventive concept as defined by the appended claims and their equivalents.

I claim:

1. An apparatus for determining a position of an object in a beating heart, comprising:
a sensor adapted to be connected to the object;
an imaging device which acquires images of the beating heart;
an electrocardiograph which produces a real-time electrocardiogram of the beating heart; and
a control unit which processes the acquired images of the beating heart and produces a moving image of the beating heart and which synchronizes the moving image with the electrocardiogram;
wherein a position of the sensor is registered with respect to the synchronized moving image by touching the sensor to landmarks within the heart and indicating a position of the landmarks on the synchronized moving image;
and wherein the control unit tracks the position of the sensor and indicates the position of the sensor on the synchronized moving image.

2. The apparatus according to claim 1, wherein the landmarks comprise electrical landmarks corresponding to known anatomic positions.

3. The apparatus according to claim 1, wherein the landmarks comprise positions at which pressure changes and which correspond to known anatomic positions.

4. The apparatus according to claim 1, wherein the acquired images comprise three dimensional images and the moving image comprises a three dimensional moving image.

5. The apparatus according to claim 1, wherein the acquired images comprise two dimensional images and the moving image comprises a two dimensional moving image.

6. A method for registering a position of a sensor inserted in a beating heart with respect to a moving image of the beating heart, comprising:
touching the sensor to a wall of the beating heart so as to cause the sensor to move with the wall of the beating heart throughout a beating cycle of the beating heart;
collecting positional coordinates of the sensor with each beat to define a beating structure; and
matching the defined beating structure with the moving image of the beating heart;
wherein the moving image of the beating heart is produced based on previously acquired images,
wherein the sensor is touched to the wall of the beating heart at points at electrical landmarks within the heart which correspond to known anatomic positions and the beating cycle is defined by indicating the anatomic positions on the three dimensional moving image of the beating heart.

7. The method according to claim 6, wherein the moving image comprises a three dimensional moving image.

8. The method according to claim 6, wherein the moving image comprises a two dimensional moving image.

9. A method for registering a position of a sensor inserted in a beating heart with respect to a moving image of the beating heart, comprising:
touching the sensor to a wall of the beating heart so as to cause the sensor to move with the wall of the beating heart throughout a beating cycle of the beating heart;
collecting positional coordinates of the sensor with each beat to define a beating structure; and
matching the defined beating structure with the moving image of the beating heart;
wherein the moving image of the beating heart is produced based on previously acquired images,
wherein the sensor is touched to the wall of the beating heart at points at which pressure changes and which correspond to known anatomic positions and the beating cycle is defined by indicating the anatomic positions on the three dimensional moving image of the beating heart.

10. The method according to claim 6, wherein the moving image comprises a three dimensional moving image.

11. The method according to claim 6, wherein the moving image comprises a two dimensional moving image.
